(19) United States
(12) Patent Application Publication
(10) Pub. No.: US 2026/0306004 A1
(43) Pub. Date: Oct. 8, 2026

(54) METHOD AND DEVICE FOR MONITORING AND OPTIMIZING A TEMPORAL TRIGGER STABILITY BY MEANS OF A P-WAVE AND/OR T-WAVE

(71) Applicants: Xenios AG, Heilbronn (DE); Hochschule Für Technik, Wirtschaft Und Medien Offenburg, Offenburg (DE)

(72) Inventor: Matthias Heinke, Rudolstadt (DE)

(21) Appl. No.: 19/482,553

(22) PCT Filed: May 8, 2024
(86) PCT No.: PCT/EP2024/062671

§ 371 Date: Nov. 7, 2025

(30) Foreign Application Priority Data
May 10, 2023 (DE) 10 2023 001 891.0

(51) Int. Cl. A61B 5/364; A61B 5/352; A61B 5/353; A61B 5/355; A61B 5/36; A61M 1/16; A61M 1/36

(52) U.S. Cl. CPC A61B 5/364; A61B 5/352; A61B 5/353; A61B 5/355; A61B 5/36; A61M 1/1601; A61M 1/36; A61M 2205/50; A61M 2230/005; A61M 2230/04

(57) ABSTRACT

The present disclosure relates to devices for monitoring and optimizing a temporal trigger stability of an extracorporeal circulatory support as well as control units for extracorporeal circulatory support, comprising such a device and corresponding methods. Accordingly, a method for monitoring and optimizing a temporal trigger stability of an extracorporeal circulatory support is suggested, comprising: receiving an ECG signal of a supported patient over a predefined time period; determining P-waves, R-waves, and T-waves from the received ECG signal; and determining a predefined trigger signal from the ECG signal of a current cardiac cycle, taking into account at least one detected P-wave and/or at least one detected T-wave.

### CROSS-REFERENCE TO RELATED APPLICATIONS

The present application is the national stage entry of International Application No. PCT/EP2024/062671, filed on May 8, 2024, and claims priority to Application No. DE102023001891.0, filed in the Federal Republic of Germany on May 10, 2023, the disclosures of which are expressly incorporated herein in their entirety by reference thereto.

### TECHNICAL FIELD

The present disclosure relates to methods for monitoring and optimizing a temporal trigger stability of an extracorporeal circulatory support as well as corresponding devices.

### BACKGROUND

In order to stabilize a patient's condition in the event of cardiac pump failure or failure of the cardiac pumping function extracorporeal circulatory support systems have been developed, which provide mechanical support. They can be quickly connected to the circulatory system. They can improve blood flow and perfusion of the organs, including the coronary arteries, and prevent a hypoxic state. For example, a blood pump can be connected to a venous access via a venous cannula and to an arterial access via an arterial cannula for respectively suctioning and pumping blood. A blood flow can be provided from a side with a low pressure, for example via an oxygenator, to a side with a higher pressure, thereby supporting the patient's circulation.

Measurement signals from an electrocardiogram (ECG) can be recorded and used to control the extracorporeal support, enabling that corresponding characteristic amplitudes for different phases of the cardiac cycle may be determined. For example, an R-wave being characteristic of the systolic phase of the cardiac cycle can usually be easily distinguished from other phases of the cardiac cycle. The R-wave can be used in the form of an R-trigger and with a predefined latency to control a blood pump in a successive diastolic phase.

Extracorporeal circulatory support can be provided for both spontaneous and stimulated cardiac rhythms. A stimulated heart rhythm can involve excitatory electrical stimulation of the heart by means of a pacemaker (PM), implantable cardioverter/defibrillator (ICD), cardiac resynchronization therapy (CRT) or non-excitatory electrical stimulation of the heart by means of cardiac contractility modulation (CCM) as well as the combination of excitatory and non-excitatory electrical stimulation of the heart. In all cases, cardiac arrhythmias, for example, can occur during the course of support, which can influence the ECG signal. For example, atrio-ventricular conduction disturbances, ventricular bundle branch block or even bradycardic or tachycardic phases of the heart rhythm can distort the ECG signal to such extent that an electrophysiological or pathophysiological change occurs in the ECG signal. This can significantly change the course of the ECG signal. The detection of an R-wave required for circulatory support can therefore be impaired or made more difficult.

Accordingly, uncertainty may arise with regard to correct detection, especially if the R-wave is not clearly recognizable in the ECG signal and/or cannot be clearly detected due to the fixed setting of the parameters. Consequently, an R-wave may potentially not be detected or may be detected at a time that does not coincide with the actual physiological time of depolarization of the ventricles. This significantly impairs extracorporeal circulatory support, as a support pulse may not be administered or may not be administered at the intended time during the diastolic phase of the heart.

Furthermore, stimulation-related disturbances of the ECG signal can occur if cardiac stimulation of the patient is provided in addition to extracorporeal circulatory support. Stimulation pulses can, for example, be administered within the QRS complex of a respective cardiac cycle, possibly with different stimulation amplitude and stimulation pulse duration and stimulation form, for example also several positive and negative stimulation pulses. In this case, no corresponding R-wave can be detected or an amplitude change in the ECG signal is inadvertently detected as an R-wave. Also, in these situations, extracorporeal circulatory support cannot be provided with the desired stability required for the patient.

Accordingly, there is a need to improve the verification of the temporal trigger stability of an extracorporeal circulatory support, e.g., under different physiological and/or clinical conditions, and to optimize the temporal electrical trigger stability during circulatory support.

### SUMMARY

The present disclosure relates to an improved verification and optimization of the temporal trigger stability of an extracorporeal circulatory support.

In one aspect, a method for monitoring and optimizing a temporal trigger stability of an extracorporeal circulatory support includes:
receiving an ECG signal of a supported patient over a predefined period of time;
determining P-waves, R-waves, and T-waves from the received ECG signal; and
determining a predefined trigger signal from the ECG signal of a current cardiac cycle, taking into account at least one detected P-wave and/or at least one detected T-wave.

Typically, for extracorporeal circulatory support systems, only one R-wave is detected from the respective cardiac cycle. At best, an additional signal of the QRS complex comprising the R-wave is detected. As described above, this results in uncertainty regarding the correct detection of an R-wave, e.g., when cardiac arrhythmia occurs, which affects the ECG signal. For example, an R-wave can be detected for a wrong time point. In this case, an amplitude change is detected that does not physiologically correspond to the R-wave. Alternatively, it may not be possible to detect an R-wave in the ECG signal. Furthermore, two R-waves can be detected with only one ventricular depolarization, for example in case of a pronounced delta wave at the beginning of the QRS complex in Wolff-Parkinson-White syndrome and intermittent right and left bundle branch blocks. Control of extracorporeal circulatory support hence cannot be ensured with the desired reliability and precision.

By taking into account an additional P-wave and/or T-wave, a further factor is taken into account when determining the trigger signal, which significantly improves the reliability and precision of a correct detection of the trigger signal. For example, a successive sequence of a P-wave, R-wave and T-wave within a cardiac cycle already indicates that the R-wave was detected between the P-wave and the T-wave. In this case, it can be ruled out that an artifact, i.e., a wave outside the P-T time interval, is inadvertently interpreted as an R-wave.

Furthermore, the detection of a P-wave indicates, for example, that an atrial depolarization has occurred. An additional and inverted P-wave after an R-wave can, for example, indicate retrograde conduction between the ventricle and atrium during ventricular stimulation. However, an inverted P-wave before an R-wave may indicate a left atrial rhythm with possible sinus bradycardia. Atrial fibrillation, in which a P-wave is typically not detected, can regularly be ruled out. If a P-wave can be detected, it can be assumed that a detected R-wave was triggered due to conduction in the AV node.

It can therefore not be assumed that a signal detected as an R-wave was triggered by a replacement rhythm.

Detection of the T-wave can also serve to confirm a previous excitation. The detection of a T-wave and the detection of two R-waves in one cardiac action can indicate bundle branch block due to a broadening of the QRS complex, for example by a pronounced delta wave, and lead to so-called “double counting”. In the absence of a T-wave, but if an R-wave is detected, an ineffective right and/or left ventricular stimulation impulse may be present, for example. In this case, the detected R-wave does not correspond to a depolarization of the ventricle. In this case, the R wave should not be used as a trigger signal.

On the other hand, a T-wave that has been determined for a current cardiac cycle may e.g. confirm the validity of a determined trigger signal in the form of a P-wave or an R-wave. For example, initially an R-wave may be determined as a trigger signal but may only finally serve as a trigger signal, if a successive (normal) T-wave was determined in the current heart cycle. In such a case, based on the R-wave, a latency for an actuation signal of the extracorporeal circulatory support may be defined or provided to, e.g., activate a blood pump in the subsequent diastolic phase, wherein the actuation signal may only be output, if the T-wave has been detected in the current cardiac cycle. For example, the actuation signal may then be output directly after the T-wave or, alternatively, already in case of a detected inclination at the beginning of a T-wave. Alternatively, the T-wave itself may also be determined as a trigger signal, for example, if a P-wave and/or an R-wave has been previously detected in the current cardiac cycle and a corresponding latency after the P-wave or R-wave for the actuation signal coincides with the T-wave. Thereby, an actuation of the blood pump of the extracorporeal circulatory support may, e.g., coincide directly with the start of a diastolic phase or with the end of a systolic phase.

In this way, the detected P-wave and/or T-wave can be used to automatically optimize and, if necessary, adjust trigger stability, for example by accordingly adjusting the control of the circulatory support. Any potential variability of the ECG signal can be detected and taken into account using the detected P-wave and/or T-wave. If it is determined that no trigger signal could be determined or if it is determined from the detected P-wave and/or T-wave that a certain trigger signal may be erroneous, a corresponding signal or warning signal can be output, for example.

However, if based on the detected P-wave and/or T-wave it is confirmed that the trigger signal can be determined or that a correct trigger signal has been determined, an actuation signal and/or control signal for the extracorporeal circulatory support can be output based on the trigger signal, e.g., with a predefined delay time or latency. It can also be provided that an input or confirmation can be requested from the medical staff.

The predefined period of time may be defined by the treatment duration or a (periodic) portion of the treatment duration, such that the receiving of the ECG signals, e.g., occurs continuously during a treatment of a patient. The period of time may also include at least the current cardiac cycle and at least one preceding cardiac cycle, e.g., at least two to five preceding cardiac cycles. The received ECG signals may be recorded or temporarily stored for a predefined number of cardiac cycles, for example in a memory, to facilitate the determining of the trigger signal and/or a potentially required evaluation or processing of the ECG signal. A rolling cache for successive cardiac cycles may be provided, such that the current cardiac cycle and the predefined number of preceding cardiac cycles are always and continuously available.

The P-wave and/or the T-wave can be detected for the current cardiac cycle and/or for at least one preceding cardiac cycle, e.g., at least or exclusively the immediately preceding cardiac cycle.

In some embodiments, the P-wave is detected at least for the current cardiac cycle. This also enables immediate confirmation or adjustment of the trigger signal to be determined for the current cardiac cycle. In other words, high-resolution monitoring and, if necessary, adjustment of the trigger signal to be determined can be provided. As described above, a T-wave can also provide feedback for the current cardiac cycle with regard to a trigger signal that has already been determined or output. For example, this provided by an end of the T-wave, which coincides with the end of the systolic phase or the beginning of the diastolic phase, indicating a time of potential administration of a support pulse.

In this way, it can be determined, for example, whether the trigger signal was detected at the correct time or whether a delay time for the support pulse was set correctly.

Alternatively, or additionally, a P-wave and/or a T-wave can also be detected for one or more preceding cardiac cycles. This allows any changes in the ECG signal curve to be detected before the current cardiac cycle. In this way, the accuracy of the P-wave and T-wave detection and the determining of the trigger signal for the current cardiac cycle can be further improved. Changes in the T-wave for the current cardiac cycle can also be taken into account in this way. If there is no T-wave (and/or P-wave) from a previous cardiac cycle, for example, a warning signal can be issued.

Accordingly, it may be provided that a P-wave is at least determined for the current cardiac cycle and a T-wave is at least determined for the directly preceding cardiac cycle. Thereby, it may already be determined from the previous cardiac cycle, if an expected repolarization or a common sequence of the preceding cardiac cycle has occurred. Furthermore, it is ensured that no overlap between the P-wave of the current cardiac cycle with the preceding T-wave is present. In this manner the validity of a determined P-wave and/or a subsequently determined R-wave may be improved or confirmed, such that the P-wave and the R-wave of the current cardiac cycle may be determined as a trigger signal.

Furthermore, ventricular repolarization can be determined and monitored by detecting a microvolt T-wave alternans over several cardiac actions using signal averaging of the T-waves and used to optimize the support pulse. In this way, trigger problems during elevation or depression of the ST segment can be detected and eliminated.

In addition to the detection of the P-waves, R-waves and T-waves in the temporal domain, learning-based methods of biosignal processing and biosignal analysis can be applied with the aid of a machine learning model such as linear regression, support vector regression, neural network, support vector machine, nearest neighbor, feed-forward neural network (FFNN), recurrent neural network (RNN) and long short-term memory (LSTM). This can further increase the temporal trigger stability during extracorporeal circulatory support. With learning-based methods, statistical parameters such as mean value, median, standard deviation, percentiles, skewness and kurtosis can be determined. Furthermore, parameters in the frequency range can be determined for the P-wave, T-wave and R-wave.

The ECG signal corresponding to the respective P-wave and/or T-wave can be statistically evaluated and, for example, be averaged or normalized for the corresponding cardiac cycles. In this way, small changes in the ECG signal course can be weighted less heavily. It is also possible for the P-wave and/or T-wave to be detected (e.g., exclusively) for an immediately preceding cardiac cycle, for example. As a result, any variations in the ECG signal can be taken into account in real time for the current cardiac cycle, in a similar way to the detection for the current cardiac cycle.

In some embodiments, the trigger signal is determined based on a P-R time interval between the P-wave and the R-wave of a respective cardiac cycle. The determining of the predefined trigger signal and/or the predefined trigger signal to be determined can be adjusted when a threshold value predefined for the P-R time interval is exceeded. In other words, in case the threshold value is exceeded the type of detection may be adjusted e.g. by adjusting parameter for the detection such as minimums, maximums, inclinations, and/or time intervals for the predefined trigger signal, for example, the R-wave. The predefined trigger signal itself may also be adjusted, such that for a current cardiac cycle or a subsequent cardiac cycle a P-wave or T-wave is to be determined as a trigger signal instead of an R-wave that has been determined so far. The type of amplitude change that is characteristic for a cardiac cycle phase, which is to be determined, may hence be adjusted.

As described above, the detection of a P-wave can already be beneficial in itself. For example, an atrial fibrillation condition can be detected or regularly excluded. In addition, it can be determined whether an atrial fibrillation occurs intermittently. In this way, any replacement rhythm that may occur and the associated lower heart rate can be taken into account. Furthermore, an analysis of the morphology of the P-wave can contribute to differentiate between, for example, sinus rhythm and left atrial heart rhythm as well as retrograde conduction between the ventricle and atrium.

However, the P-R time interval can also be used to detect other heart diseases affecting excitation such as atrio-ventricular conduction disorders, ventricular bundle branch block and/or additional conduction pathways between the atrium and ventricle as so-called Kent pathways with increased certainty. For example, an atrioventricular block can be present if the corresponding threshold value is exceeded. If the interval is within or falls below the corresponding threshold, an open Wolff-Parkinson-White syndrome (WWP syndrome) with antegrade additional conduction between the atrium and ventricle via a Kent fiber may be present and cause a change and prolongation of the QRS complex. Furthermore, a combination of maximum pre-excitation of the QRS complex via the Kent fiber and a Mahaim fiber, which may lie between the atrioventricular node (AV node) and right ventricular myocardium, can alter the electrocardiographic time intervals. A prolonged P-R time interval may on the one hand cause the P-wave to merge with the preceding T-wave. This can render it more difficult to detect the P-wave or the T-wave without a corresponding adjustment of the detection. On the other hand, such an extension of the P-R time interval can also be accompanied by a broadening or electrophysiological change in the QRS complex. As a result, an R-wave may not be detected. However, if the R-wave is to be determined as a trigger signal, the extension of the P-R time interval can be accompanied by a lower stability of the trigger signal. Taking the P-R time interval into account is also advantageous because the P-R time interval describes the beginning of the systolic phase of the cardiac cycle. If an extension of the P-R time interval or an exceeding of the corresponding threshold value is detected, another trigger signal to be determined, such as e.g. a P-wave instead of an R-wave determined so far, can be specified for the current cardiac cycle, or at least for the subsequent cardiac cycle, in order to ensure the desired trigger stability.

The P-R time interval is specified above and below as a criterion. Alternatively, a P-Q time interval can also be taken into account if the Q-wave is to be (unambiguously) detected in the ECG signal in addition to the R-wave.

In some embodiments, the detection of the P-wave, R-wave and/or T-wave for the current cardiac cycle is adjusted as soon as a threshold value predefined for the P-R time interval is exceeded for at least one previous cardiac cycle. As described above, the P-wave can merge into the T-wave if the P-R time interval is extended. Detection of the P-wave and/or T-wave may be more difficult. There may also be a broadening of the QRS complex or an electrophysiological change in the QRS complex. For example, the amplitude of the R-wave may be reduced or the R-wave may even be absent. The adjustment of the detection enables to adjust the detection for the current cardiac cycle to the heart disease pattern.

For example, the detection can be adjusted so that different slope values to be detected or detection times can be provided. A change can also be made to a respective bandpass filter. An alternative ECG lead can be selected for the respective detection. The ECG lead can, for example, be spatially and/or anatomically separated from the previously used ECG lead. Several ECG leads can also be taken into account for signal optimization. The corresponding ECG signals can, for example, be processed and can be averaged using “signal averaging”.

The threshold value for the P-R time interval can be at least 120 ms, e.g., in the absence of an antegrade conduction via a Kent path. This is because the threshold value for the P-R time interval is shortened to a few milliseconds in the case of antegrade conduction via the Kent pathway. The end of the P-wave and the beginning of the QRS complex can also be superposed with a 0 ms R-Q time interval. A threshold value for the P-R interval with antegrade conduction via the Kent pathway can be between 0 and 120 ms, depending on the characteristics of the delta wave and the selected ECG lead. In this way, it can be determined whether the P-R time interval is non-physiological with increased probability and corresponds, for example, to a first-degree AV block. In some embodiments, the threshold value for the P-R time interval is between 150 ms and 400 ms, e.g., the threshold value can be between 200 ms and 300 ms. Such a threshold value can, for example, correspond to a first-degree or second-degree AV block. If the threshold value is exceeded, there may be a change in the QRS complex and impaired detection of the R-wave.

The analysis of the P-R time intervals and the morphology of the P-wave can be used for the evaluation of AV block I°, AV block II° type 1 “Wenckebach”, AV block II° type 2 “Mobitz” and AV block III° as well as for sinus atrial block. Such an atrioventricular biosignal analysis can further support the temporal stability of R-triggering.

Without atrioventricular conduction disturbances and without antegrade conduction between the atrium and ventricle via an additional Kent pathway, the P-Q interval can lie in the range of 120 ms to 200 ms and the threshold value can be selected accordingly. The threshold value can also be dependent on the cardiac cycle duration and/or the heart rate. This is because a shorter P-Q time interval is to be expected at a higher heart rate in the physiological state. Accordingly, the threshold value for the P-Q time interval can correspond to at least 20 percent of the cardiac cycle duration, e.g., at least 30 percent or at least 35 percent. The threshold value for the P-Q time interval can also be less than 200 ms, for example without atrioventricular conduction disturbances, but should have a higher value than the respective patient-specific (patho)physiological value, e.g., be increased by at least 10% compared to the (patho)physiological value of the patient (e.g. 180 ms compared to a (patho)physiological patient value of 160 ms). Any changes in the P-R time interval due to the occurrence of a bradycardic or tachycardic state can be taken into account in this way.

In case of ventricular stimulation with pacemakers, retrograde conduction from the ventricle to the atrium can also occur, wherein an additional P-wave is generated with a modified morphology of the P-wave, for example with an inhibition of the P-wave. This retrograde P-wave can influence the pacing behavior of a dual-chamber pacemaker and the R-triggering. The R-P time interval with a retrograde conduction time of 200 ms to 300 ms, for example, can lead to premature ventricular stimulation and thus to a tachycardic heart rhythm with a corresponding effect on the hemodynamics and to fluctuating R-R time intervals. The detection of P-waves and determining of P-R and R-P intervals thus advantageously enables the evaluation of intermittent or permanent retrograde conduction between the ventricle and atrium for the optimization of the temporal stability of R-triggering.

For example, in order to give less weight to individual, rare and/or less prominent “outliers”, the P-R time interval and/or R-P time interval can also be averaged for at least two cardiac cycles. Accordingly, the time intervals between the respective P-waves and R-waves of the respective cardiac cycles are averaged, such that at least two P-R time intervals (R-P time intervals) are averaged. In some embodiments, P-R time intervals and/or R-P time intervals of successive cardiac cycles are averaged.

For example, a mean value with a standard deviation or a median based on a histogram can be determined for several cardiac cycles. In this manner, the detection and/or the trigger signal to be determined can be adjusted, for example, only if the mean value or the standard deviation or the median exceeds a corresponding statistical threshold value. Advantageously, minor and/or temporary changes hence do not immediately trigger a corresponding parameter adjustment. Any influence on the circulatory support resulting from an adjustment can hence be accordingly adopted or leveled out.

However, the number of P-R time intervals and/or R-P time intervals to be averaged can be kept low and be limited to e.g. two to five cardiac cycles. Only when the threshold value is exceeded can it be validated whether a pathological change and/or retrograde conduction from the ventricle to the atrium must actually be assumed. At the same time, a rapid adjustment of the detection and/or the trigger signal to be determined can be provided.

In some embodiments, the P-R time interval and/or R-P time interval are compared for at least two successive cardiac cycles. The determining of the predefined trigger signal and/or the predefined trigger signal to be determined can be adjusted upon a successive extension of the P-R time interval and/or R-P time interval.

A successive extension of the P-R time interval can occur, for example, due to a Wenckebach block. A successive extension of the R-P time interval can occur, for example, due to retrograde Wenckebach behavior. For example, the P-R time interval may be prolonged for two to three consecutive cardiac cycles. Under such circumstances, conduction fails; an R-wave or excitation of the ventricles does not occur. In the case of a Wenckebach block, for example, every second or every third P-wave can also be transmitted. This pathophysiology can mean that the detection of the R-wave is to be omitted and the P-wave can be used as a trigger signal instead.

Such a successive extension can occur, e.g., if the corresponding threshold value is also exceeded. The extension can, for example, be determined using an absolute difference value or in relation to the previous cardiac cycle. For example, the extension or prolongation can be 50 ms to 100 ms and/or at least 20 percent.

The trigger signal can alternatively or additionally be determined based on an R-T time interval between an R-wave (or several P-waves in the case of retrograde conduction between the ventricle and atrium) and a T-wave of a respective cardiac cycle. In some embodiments, the determining of the predefined trigger signal and/or the predefined trigger signal to be determined is adjusted when a threshold value predefined for the R-T time interval is exceeded.

Taking the R-T time interval or, by analogy, the Q-T time interval into account also enables to detect cardiac diseases affecting the excitation, such as ventricular excitation regression disorders with and without bundle branch block, with increased detection reliability. For example, an extension of the R-T time interval can occur due to a fusion of the T-wave with a successive P-wave and/or due to a broadening of the QRS complex. These changes in the QRS complex can be caused, for example, by additional conduction pathways between the atrium and ventricle, so-called antegrade conduction Kent pathways. The Kent pathways can be located at the level of the valve of the right and/or left heart and can conduct continuously or intermittently antegrade or retrograde. Antegrade conduction via a left cardiac lateral Kent pathway, for example, results in the so-called delta wave with corresponding broadening of the QRS complex and reduction of the P-R time interval. In Wolff-Parkinson-White syndrome, the so-called WPW syndrome, premature excitation of the left ventricle with widening of the QRS complex occurs due to the antegrade, additional conduction via the Kent pathway. A reduced P-R time interval can indicate an overt WPW syndrome with widening of the QRS complex.

Exceeding the threshold value for the R-T time interval can serve as an indicator of a pathological condition and, for example, enable an early adjustment of the acquisition and/or the trigger signal to be determined. Trigger stability can therefore also be significantly improved when cardiac arrhythmias occur. As described above, the detection and consideration of the T-wave can not only improve trigger stability as such, for example by allowing an excitation to be checked. It also enables a more precise adjustment of the trigger signal to a given pathophysiological condition by means of the R-T time interval.

In some heart diseases or cardiac arrhythmias, a shortening of the T-wave may occur. Although the threshold value can represent an upper time limit, the threshold value can also include a lower time limit. For example, the threshold value can be defined by a range which, under normal circumstances, should neither be fallen below nor exceeded.

In some embodiments, it is provided that the detection of the P-wave, R-wave and/or T-wave for the current cardiac cycle is adjusted, if a threshold value predefined for the R-T time interval is exceeded in at least one previous cardiac cycle.

The T-wave occurs at the end of the systolic phase and is subsequent to the P-wave and R-wave. This enables to optimize the detection for a subsequent cardiac cycle. For example, a P-wave of a current cardiac cycle can be fused with the T-wave of the immediately preceding cardiac cycle if the R-T time interval is extended. Accordingly, a more precise detection of the P-wave may be required, for example by means of a parameter change, such as a frequency range predefined for the P-wave.

In some embodiments, the threshold value for the R-T time interval is at least 350 ms. Similar to the described P-R time interval, non-physiological R-T time intervals, which may require an adjustment of the detection and/or the trigger signal to be determined, can hence be recognized and taken into account.

The threshold value for the R-T time interval can also be set to at least 450 ms. In this way, the occurrence of some types of cardiac arrhythmia, for example in the case of an AV block, can be detected and taken into account when determining the trigger signal. As described above for the P-R time interval, the threshold value can also be dependent on the cardiac cycle duration and/or the heart rate. It should be noted that a shorter Q-T time interval and a correspondingly shorter R-T time interval are expected at a higher heart rate in the physiological state. Thus, the threshold value for the R-T time interval can correspond to at least 35 percent of the cardiac cycle duration, e.g., at least 40 percent or even at least 45 percent.

The R-T time interval can be averaged for at least two cardiac cycles, as described above with regard to the P-R time interval.

The detection of the T-wave alone can therefore already enable the current cardiac cycle to be monitored. The R-T time interval also enables to recognize potential pathophysiological changes. Furthermore, the T-wave can also be taken into account to detect cardiac arrhythmias, for example. Thus, the determining of the predefined trigger signal, the predefined trigger signal to be determined and/or the detection of the P-wave, R-wave and/or T-wave for the current cardiac cycle can, e.g., be adjusted if an amplitude of the T-wave for at least one previous cardiac cycle comprises a negative value or is negative overall.

In some embodiments, an adjustment of a subsequent cardiac cycle can be provided in this way. If the amplitude is negative or significantly flattened, this finding may indicate a bundle branch block of the cardiac conduction system.

In some embodiments, the trigger signal is determined based on a QRS time interval between a Q-wave and an S-wave of a respective cardiac cycle. The determining of the predefined trigger signal and/or the predefined trigger signal to be determined can be adjusted when a threshold value predefined for the QRS time interval is exceeded. Alternatively, or additionally, the detection of the P-wave, R-wave and/or T-wave for the current cardiac cycle can be adjusted if a threshold value predefined for the QRS time interval is exceeded for at least one previous cardiac cycle.

Due to cardiac arrhythmias and, e.g., in the case of an atrio-ventricular bundle branch block, the QRS complex may be widened or lengthened or the ECG signal curve within the QRS complex may be significantly altered. For example, the R-wave may not occur or may occur with a significantly lower amplitude value than usual. If the threshold value for the QRS time interval is exceeded, this circumstance—for example due to the presence of a bundle branch block—can prevent the determining of a trigger signal within the QRS complex, for example an R-wave. In this case, an alternative amplitude change, e.g., a P-wave, can be determined as a trigger signal. Any parameter values required for the detection can also be adjusted in order to optimize the detection for subsequent cardiac cycles, For example, a frequency range set for the detection of the QRS complex can be adjusted and/or a lower threshold value can be set for the detection of an R-wave.

The threshold value for the QRS time interval can be at least 120 ms. In this way, physiological values, which are, e.g., between 90 ms and 110 ms, can be excluded and a cardiac arrhythmia can be detected or confirmed.

The P-R time interval, R-T time interval or QRS time interval described above can each individually improve the trigger stability and thus the extracorporeal circulatory support. In some embodiments, however, at least two of these time intervals are taken into account when determining the trigger signal. In this way, any cardiac arrhythmia can be detected more accurately and even anticipated for the control of the extracorporeal circulatory support. In some embodiments, the P-R time interval, R-T time interval and QRS time interval are taken into account in combination. The time intervals can form an ECG signature together.

The detection of the P-wave, R-wave and T-wave and the determining of the respective time intervals occur automatically, for example by implementing the method on a computer. For example, the procedure can be carried out by a processor of an ECG device or by a control unit for extracorporeal circulatory support. In this way, an adjustment of the detection can also occur automatically or be provided or suggested. Thereby, the trigger stability can be continuously monitored and improved.

A threshold value for one or more time intervals within a respective cardiac cycle and/or the predefined trigger signal can be based on a pathological ECG signature. For example, a threshold for a P-R interval may be provided for a cardiac arrhythmia, such as in the case of a first degree AV block. In such circumstances, it may also be envisaged that, for example, a P-wave rather than an R-wave is defined as the trigger signal. The pathological signature characterizing a certain heart disease pattern can therefore, if necessary, specify an ECG signal curve or course or the duration of one or more specific time intervals.

The pathological ECG signature can be preset and/or be determined on the basis of time intervals detected in preceding cardiac cycles for the current cardiac cycle. For example, a pathological ECG signature can be preset for the extracorporeal circulatory support of a patient so that trigger stability for circulatory support can already be optimized initially. For example, the ECG signal of the supported patient can be compared with corresponding threshold values for specific time intervals, such as the P-R time interval, with the set or installed pathological ECG signature.

However, the pathological ECG signature can also be determined using the ECG signals from preceding cardiac cycles. Thus, corresponding threshold values can be determined by “online” analysis of the ECG signals during a current circulatory support, for example by means of a statistical evaluation and learning-based methods in the time and frequency domain. For example, time intervals, such as the P-R time interval and/or the R-T time interval, of successive cardiac cycles can be detected and determined and defined as a histogram or mean value or median with standard deviations or percentiles as well as skewness and kurtosis (curvature).

For example, ECG signals can be evaluated “online” while an extracorporeal circulatory support is being performed and optionally be used for learning-based methods. The ECG signal can be received immediately after acquisition or detection, e.g., continuously. The initially acquired data can be validated and annotated during the detection of the ECG signal and the extracorporeal circulatory support. They can then be used to determine one or more thresholds for successive cardiac cycles. The data can also be evaluated or analyzed with a time delay during extracorporeal circulatory support to enable a “near-line” analysis. Learning-based methods can include linear regression, Support Vector Machine (SVM), Artificial Neural Network, Feed-Forward Neural Network (FFNN), Recurrent Neural Network (RNN) and Long Shorth-Term Memory (LSTM).

The ECG signal and the corresponding data can be at least partially or completely buffered or cached, e.g., for a predefined time range. In this way, for example, an evaluation unit can take into account the course of the patient's own ECG signal when determining the trigger signal and/or a respective threshold value. This can increase or improve the accuracy of the monitoring and, if necessary, the placement of the trigger signal.

Furthermore, a trigger signal to be determined can be predefined on the basis of the preceding cardiac cycles. For example, a P-wave or an R-wave can be specified as a trigger signal, provided that the P-P time intervals or R-R time intervals detected in a minimum number of (optionally consecutive) cardiac cycles could be detected consistently and with little deviation.

Determining of the pathological ECG signature based on preceding cardiac cycles can also be provided in addition to a preset ECG signature in order to enable monitoring of the trigger stability immediately after determining and at the same time enabling immediate adjustment to the patient-specific circumstances.

Finally, the time intervals detected from the preceding cardiac cycles can also be compared with an “offline” evaluated data set with ECG signals from supported patients who have pathological findings in common with the patient to be treated. The ECG signature that shows the best possible match or fit with an ECG signal from the data set can be predefined as a parameter, wherein learning-based methods can also be applied to determine the matching.

The data set stored “offline” can, for example, be annotated for the P-waves, R-waves and T-waves in order to improve the accuracy of the detected time intervals. The annotated data can be used to specify statistically optimized threshold values for the time intervals used, which correspond to the respective pathology. Alternatively, or additionally, suitable frequency ranges for the P-waves, R-waves and T-waves and/or suitable amplitude values can be predefined so that a comparison of the patient's ECG signal with the data set enables an increased matching.

For each cardiological pathology, a time interval range, frequency range and/or amplitude range and/or a trigger signal to be determined within a defined time interval, e.g. the R-T time interval, can be specified or predefined. The time interval to be used can be selected, e.g., from the P-R time interval, the R-T time interval and/or the QRS time interval.

To improve the accuracy of the detection of the amplitude changes in the ECG signal, the P-wave, the R-wave and/or the T-wave can be detected after passing a bandpass filter. A frequency range is, e.g., between 5 Hz and 20 Hz for the P-wave, e.g., between 10 Hz and 25 Hz for the R-wave, and/or, e.g., between 1 Hz and 5 Hz for the T-wave. In some embodiments, the detection of the respective amplitude changes can be significantly improved by processing the ECG signal using the above frequency ranges. If the detection needs to be adjusted during the course of the circulatory support, for example as a result of a threshold value for a specific time interval within the cardiac cycle being exceeded, one or more frequency ranges provided for the detection can be adjusted accordingly.

Typically, an R-wave or R-wave spike from the respective cardiac cycle is determined for the control of an extracorporeal circulatory support, which is to serve as a suitable trigger signal. As described above, under certain pathophysiological conditions, the detection of the R-wave, for example, can occur at the wrong time or be impaired, for example because an amplitude change different than the R-wave is detected. Such circumstances can also occur, for example, if the patient is undergoing cardiac stimulation and the stimulus causes an artifact in the ECG signal. An R-wave may therefore not provide the desired or required trigger stability for extracorporeal circulatory support in certain cardiac conditions.

In this case, the predefined trigger signal can be an R-wave. An alternative or additional trigger signal such as a P-wave or T-wave can be selected by taking into account the at least one detected P-wave and/or the at least one detected T-wave.

The R-wave can hence be preset as the trigger signal. However, the trigger signal can be adjusted, for example, when a threshold value for a P-R time interval is exceeded. This allows a P-wave to be used as the trigger signal for at least one subsequent cardiac cycle, for example. In this way, it can be ensured that a trigger signal can be determined and used with sufficient stability even in the event of distortions or changes in the ECG signal,

The use of the P-wave also has the advantage that the trigger signal can even be adjusted for the current cardiac cycle using the P-R time interval of the current cardiac cycle, for example. The P-wave and the P-R time interval do not collide or overlap with a subsequent diastolic phase. A trigger signal can be provided or output—algorithmically—with a suitably predefined delay time in order to control the extracorporeal circulatory support during a subsequent diastolic phase. This occurs in this way even if an R-wave could not be detected or could not be detected with sufficient detection reliability.

In some embodiments, it is provided that—based on the determined trigger signal—a signal for the extracorporeal circulatory support is output with a delay time. The delay time can be predefined taking into account the at least one detected P-wave and/or the at least one detected T-wave.

The circulatory support signal can, for example, be a control signal for the extracorporeal circulatory support, such as for a blood pump. For example, the delay time can be adjusted when the P-R time interval and/or a QRS time interval is extended. This ensures that the signal is not inadvertently provided in an unintended cardiac cycle phase. Adjusting the delay time may prevent the extracorporeal circulatory support from delivering a support pulse during a systolic phase of the cardiac cycle.

Furthermore, it may also be provided that the delay time is automatically adjusted or automatically suggested depending on the trigger signal to be determined. This procedure can be advantageous, for example, if the trigger signal to be determined is changed, e.g. if a P-wave is or should be determined instead of an R-wave. A predefined delay time or latency may no longer be sufficient to prevent overlapping or interleaving with a systolic phase, such that the delay time is to be adjusted. An R-wave-based delay time can, for example, be extended by the P-R time interval or alternatively by a P-Q time interval.

If neither a Q-wave or P-wave nor an R-wave could be detected, the delay time can be adjusted in the event of excitation formation and/or excitation conduction disturbances and or stimulation of the heart, for example by including a hemodynamic sensor. Complex electrical and hemodynamic cardiac situations can arise for the adjustment of the delay time, which can be influenced by the electromechanical coupling. For example, electromechanical decoupling of the heart is followed by electrical excitation of the heart without mechanical contraction of the heart. However, the hemodynamic sensor can be used to provide a further alternative or a redundant system in order to enable optimized triggering even in such cases.

The delay time for the current cardiac cycle can also be determined from a detected QT time of at least one preceding cardiac cycle. The QT time can be normalized using a heart rate determined from the ECG signal.

The QT time corresponds to the period of the cardiac cycle from the beginning of the Q-wave of the QRS complex to the end of the successive T-wave. The time point of the end of the T-wave corresponds physiologically with the closing of the aortic valves and marks the end of the systole or the beginning of the diastole. The consideration of the QT duration proves to be advantageous because, according to a rule of experience, this duration also corresponds approximately to the duration of the subsequent diastole. The delay time and the duration of a support pulse can be adjusted to the desired time of providing the trigger signal in the respective cardiac cycle phase.

Normalization and/or consideration of the heart rate has the advantage that the QT time is typically dependent on the patient's heart rate. It is to be expected that a change in the QT time should be accompanied by a change in the heart rate. Conversely, it can be advantageous to determine a heart rate-optimized delay time in this way. A normalized QT time therefore already takes the variable heart rate into account. A delay time can thus be selected using the “Framingham formula” to represent the heart rate-based QT time. In this way, a (theoretical) delay time for the current cardiac cycle can be calculated using normalized values and the current heart rate. Normalized values can be based on empirical values and can, for example, be predefined using previous data sets that were previously evaluated and validated “offline”. The delay time can be calculated using the following variant of the “Framingham formula”, for example:

$$Delay ti me = 380 - 154 \times ( 1 - 60 / heart rate )$$

In the case of right ventricular pacing (RVP) of the heart, the QT time can be advantageously determined by the stimulus T-time. The RVP T-time can be used in frequency-adaptive pacemakers to control the stimulation frequency during exercise by measuring the RVP T-time.

The heart rate can be determined by the time interval between two R-waves of successive cardiac cycles or also by the time interval between two P-waves of successive cardiac cycles, i.e. by a so-called R-R interval or P-P interval. In this way, the heart rate of the current cardiac cycle can also be taken into account. The delay time can be determined and optimized for each cardiac cycle. It is corrected almost automatically if the heart rate changes. If the systolic phase shortens due to an increase in the heart rate, for example, the delay time can be adjusted accordingly. In this way, a corresponding shortening of the QT time can be taken into account when determining the delay time by increasing the heart rate.

The trigger signal can therefore be delivered with increased probability at a time that does not fall within the systolic phase. An afterload can thus be reduced or even avoided as far as possible. If the P-wave is determined as the trigger signal, a P-Q time interval or P-R time interval can be extrapolated to the delay time in addition to the heart rate-optimized delay time. For example, an automatic correction of the delay time can be provided after a change in the trigger signal.

Alternatively, a delay time can be determined using the so-called “left ventricular ejection time” (LVET) with the aid of impedance cardiography, wherein the end of the LVET time interval signifies the closure of the aortic valve.

Furthermore, a device for monitoring and optimizing a temporal trigger stability of an extracorporeal circulatory support is suggested, comprising (i) an interface for receiving an ECG signal of a supported patient over a predefined period of time, and (ii) an evaluation unit which is configured to determine P-waves, R-waves and T-waves from the received ECG signal. The evaluation unit is advantageously configured to determine a predefined trigger signal from the ECG signal of a current cardiac cycle, taking into account at least one detected P-wave and/or at least one detected T-wave. For example, the device can be configured to carry out the method described above.

The ECG signal of the supported patient can be received via an interface of the device. The ECG signal can be processed using a working memory and/or stored in a storage medium. For example, the ECG signal can be communicatively connected to the evaluation unit via the interface. The evaluation unit can comprise both a working memory and a storage medium. ECG signals can be received via the interface from various spatially separated ECG leads. Several ECG signals can also be received. The evaluation unit is therefore advantageously configured to evaluate the ECG signals in order to determine the P-waves, R-waves and T-waves as well as the trigger signal.

In some embodiments, the aforementioned device is formed as part of an ECG device. However, it can also be communicatively connectable to at least one ECG device via an interface or the interface(s) described above. In some embodiments, the device is integrated into a control unit for an extracorporeal circulatory support. The control unit can be configured to output a control signal for the extracorporeal circulatory support at a predefined time after a specific trigger signal and with a predefined delay time, taking into account the at least one P-wave and/or the at least one T-wave.

In the control unit, the device can be provided as an integrated ECG device or attached to the control unit as an ECG device. In this way, the control unit can be used independently of other components and be compact in design. In some embodiments, the ECG device is integrated in a single housing of a system for the extracorporeal circulatory support, for example in a sensor box in the form of an ECG card or an ECG module.

The control unit can also be arranged in a console, which has a user interface for entering and reading out settings of the system, e.g., parameters of the blood pump of an extracorporeal circulatory support system and/or the ECG device. For example, the console may include a touch screen and/or a display with a keyboard that can be operated by a user. The control unit can operate, actuate, control, regulate and monitor the blood pump. It enables the blood pump to be synchronized with the cardiac cycle and, e.g., the trigger signal provided by the patient.

Further advantages as well as possible embodiments and further developments of the methods have already been described in detail with regard to the control unit described above, so that the corresponding aspects will not be described again in order to avoid redundancies. However, the corresponding contents of the disclosure continue to apply to this subject matter.

### BRIEF DESCRIPTION OF THE DRAWINGS

Embodiments are explained in more detail in the following description of the Figures.

FIG. 1 shows an electrocardiographic course of an ECG signal for successive cardiac cycles in a physiological state;

FIG. 2 shows the electrocardiographic course of the ECG signal according to FIG. 1 with annotated ECG features;

FIG. 3 shows a change in the electrocardiographic course with additional antegrade conduction via a Kent pathway in a WPW syndrome;

FIG. 4 shows a change in the electrocardiographic course of atrioventricular re-entry tachycardia (AVRT) with a retrograde Kent pathway;

FIG. 5 shows a change in the electrocardiographic course similar to that shown in FIG. 4 with an intermittent right bundle branch block; and

FIG. 6 shows an embodiment of a device as described herein.

### DETAILED DESCRIPTION

In the following, embodiments are described with reference to the Figures, Here, identical, similar or similarly functioning elements in the different Figures are provided with identical reference signs, and a repeated description of these elements is partially omitted in order to avoid redundancies.

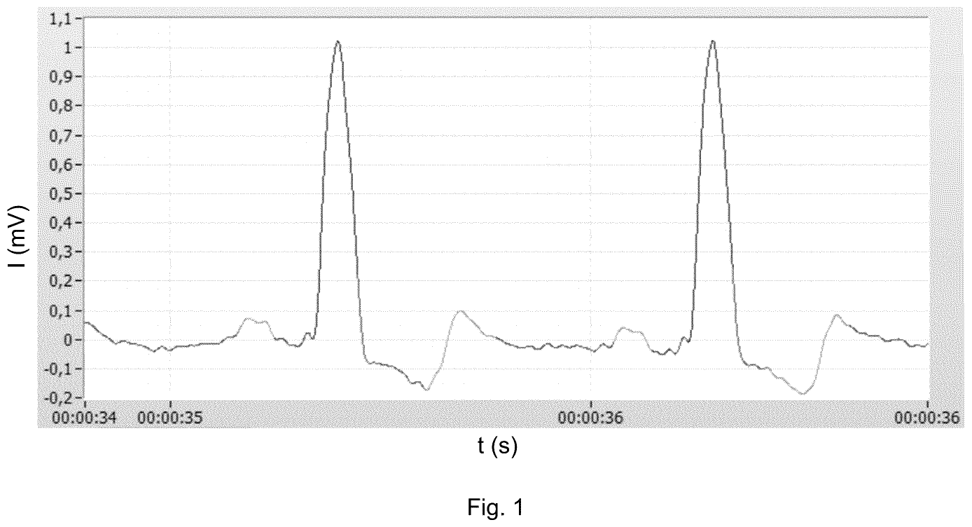

FIG. 1 shows an ECG signal course for two successive cardiac cycles over a predefined time. This is a physiological curve. The respective cardiac cycle phases are color-coded. Accordingly, each cardiac cycle begins with the depolarization of the atrium, which is known as the P-wave and is indicated by the amplitude change preceding the characteristic R-wave and having a respective current value/of about 0.1 mV.

This is followed for each cardiac cycle by a characteristic and clearly recognizable R-wave or R-spike, which respectively has an amplitude with a current value/of around 1 mV. The R-wave, which is usually easy to detect in the physiological state, is caused by the depolarization of the ventricle, which marks the beginning of the systolic phase. The R-wave is preceded by a Q-wave and followed by an S-wave, each of which has a lower negative current value. Together, these amplitude changes form the so-called QRS complex.

After the contraction of the ventricle or after the excitation induced by the R-wave, the repolarization or excitation recovery of the ventricle takes place in the T-wave, wherein the respective amplitude in the present example has a respective current value of about 0.1 mV which is colored green and has an overall positive current value.

In this example, the ECG signal was obtained from an ECG lead I for two cardiac actions in sinus rhythm. Normal atrio-ventricular conduction (AV conduction) can be observed.

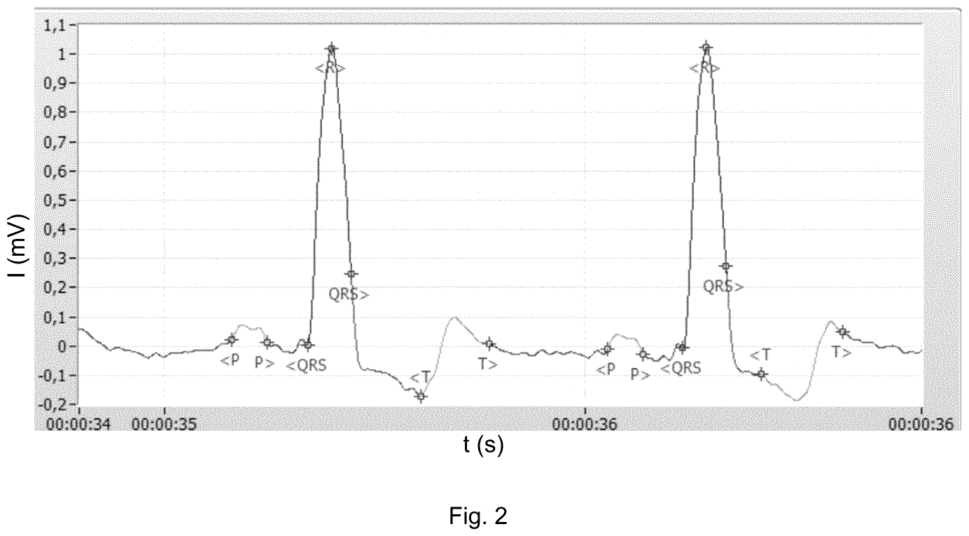

In FIG. 2, some characteristic features of the two cardiac actions according to FIG. 1 were automatically detected or determined. Based on these characteristics, specific time intervals and amplitude changes can be determined for the respective cardiac cycle. A trigger signal can be determined or, if necessary, adjusted based on a comparison with predefined threshold values.

In this example, the start and end of the P-wave, the position of the R-wave, the start and end of the QRS complex and the start and end of the T-wave were detected automatically. The detection was carried out, e.g., by means of an advantageous processing of the ECG signal using suitable bandpass filters. A bandpass filter between 10 Hz and 25 Hz was used for the QRS complex, a bandpass filter between 5 Hz and 20 Hz for the P-wave and a bandpass filter between 1 Hz and 5 Hz for the T-wave. Based on the expected frequency range and, if applicable, an expected maximum heart rate and/or an expected duration of the respective ECG feature or the respective amplitude change, the corresponding features in the ECG signal can be automatically detected in an advantageous way, for example by means of an appropriately set feature extraction.

The annotated cardiac cycles can be used to determine time intervals, e.g. the P-R time interval, the QRS time interval and the R-T time interval or Q-T time interval. In the present example, the P-R time interval is between 170 ms and 185 ms, the QRS time interval between 95 ms and 110 ms and the Q-T time interval or R-T time interval between approximately 385 ms and 435 ms. The time intervals can assume individual values for each cardiac cycle and can vary depending on the heart rate.

Based on the determined time intervals and a comparison with suitably predefined threshold values, the trigger signal to be determined and/or the detection of the P-wave, R-wave and/or T-wave can be adjusted, for example, when the respective threshold value is exceeded. In any case, a trigger signal can/could be determined with increased reliability. Thereby, a trigger stability for an extracorporeal circulatory support may be considerably improved.

If a predefined threshold value is exceeded, there may be evidence of a cardiac arrhythmia in the form of an atrio-ventricular block or a bundle branch block, for example. As described above, this can cause conduction failure. A depolarization of the ventricle may not occur or may not be adequately detected for a cardiac cycle.

For example, an R-wave with a lower amplitude, as indicated by the lowercase “r”, may occur or no R-wave may be formed at all. A double R-wave (of lower amplitude) and/or a Q-wave of lower amplitude and/or an S-wave of lower amplitude may also occur.

Examples of bundle branch block are an (in) complete right bundle branch block or left bundle branch block. These can occur intermittently or non-intermittently. An AV block can be characterized, e.g., by a double R-wave in the QRS complex, for example in the form of an M-shaped R-s-R′ curve. Such a course can be dependent on the respective ECG lead and, for example, in the case of a left bundle branch block, can be formed in ECG lead I, V5 and/or V6. A positive amplitude value can be present for the R-wave in the ECG leads I, aVI, V5, and/or V6, while a negative amplitude value for the R-wave can occur in the ECG leads III, aVR, aVF, V1, V2 and/or V3. In the case of a right bundle branch block, the S-wave can be deeper and/or wider, for example in ECG leads I, aVL, V5 and/or V6, and/or the R-wave can be higher, wider and, e.g., split, for example in ECG leads III, aVL, V1 and/or V2.

Based on the course of the ECG signal and/or respective amplitude changes within the QRS complex, forms of cardiac arrhythmia can thus be determined and taken into account when determining the trigger signal. This procedure can be carried out in addition to the consideration of the at least one P-wave and/or T-wave.

Instead of a bundle branch block or a block of the conduction system, a left anterior or left posterior hemiblock can also occur. A bifascicular block may also be present, for example a right bundle branch block with a corresponding hemiblock. As described above, these pathophysiologies can be diagnosed using the P-wave, the T-wave and/or the QRS complex and be taken into account when determining the trigger signal. Furthermore, (bradycardic or tachycardic) ventricular and/or supraventricular conduction disturbances may be present.

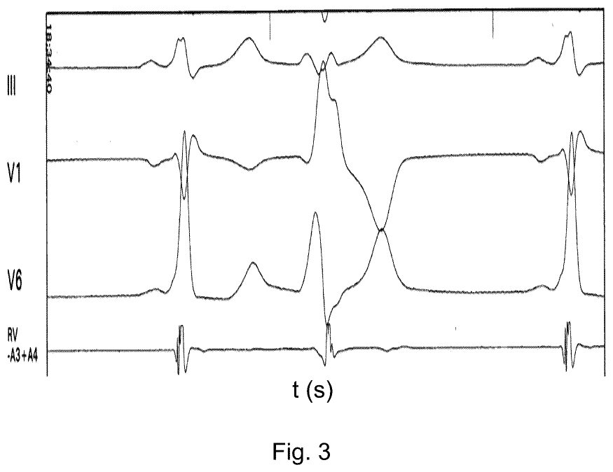

FIG. 3 shows an electrocardiogramamely surface ECG leads III, V1 and V6, with a right ventricular ECG RV34 also shown in the lower section. The upper markings define a time interval of 1000 ms in each case.

The ECG course corresponds to a pathological course for a Wolff-Parkinson-White syndrome (WPW syndrome) with additional antegrade conduction via a Kent pathway. It can be seen that the additional antegrade conduction occurs intermittently in the present case, namely during the first and third cardiac action. Accordingly, the additional premature conduction between the atrium and the ventricle results in a shortening of the P-Q time interval. A ventricular extrasystole can still be seen in the second cardiac action. In order to detect the R-wave correctly, a corresponding P-Q time interval or P-R time interval can be shortened or a corresponding threshold value can be adjusted to enable stable triggering based on a detected R-wave even in the case of such a pathological course.

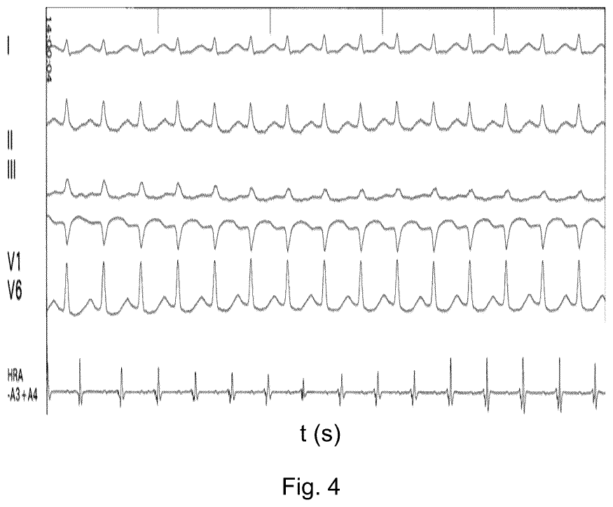

FIG. 4 shows an electrocardiogram the case of another cardiologic pathology, namely an atrioventricular re-entry tachycardia (AVRT) with a retrograde Kent pathway. As in FIG. 3, a narrow QRS complex is also present in this case. The ECG course was recorded without right bundle branch block and without left bundle branch block for the surface ECG leads I, II, III, V1, and V6, wherein a right atrial ECG HRA34 is also shown in the lower section. As in FIG. 3, the upper markings also define a time interval of 1000 ms.

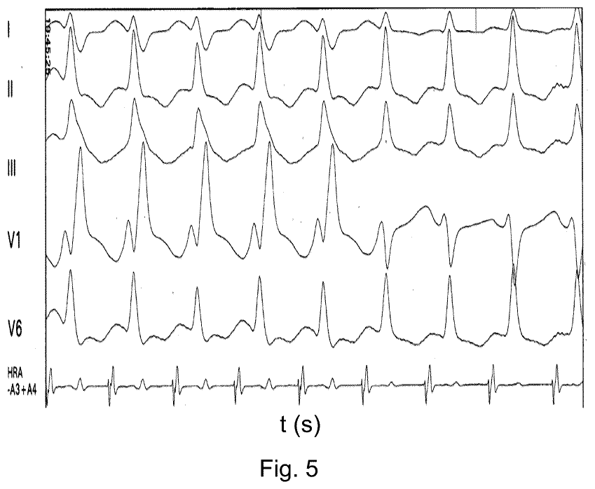

In the case of an intermittent right bundle branch block, the ECG curve changes accordingly, as shown in FIG. 5. The Figure shows how the QRS complex widens when a right bundle branch block occurs. The effect is most pronounced in ECG leads I and V1. Accordingly, when determining such a broadening of the QRS complex, not only can a pathological course be determined or confirmed, but an adjustment of the intended time intervals, for example, can also be provided in order to support or facilitate future detection of an R-wave.

QRS time intervals or frequency ranges can be analyzed in the ECG in the case of a right bundle branch block as shown in FIGS. 1 and 2.

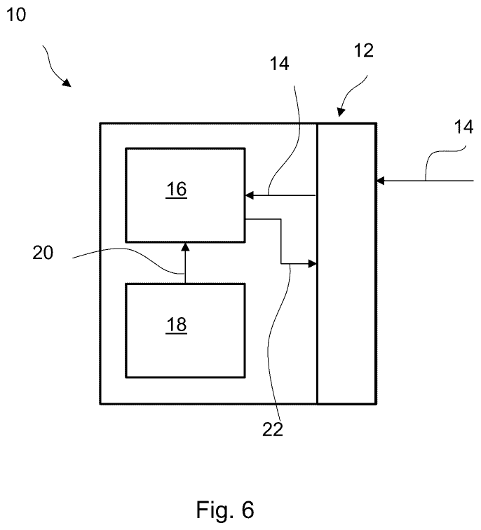

FIG. 6 schematically shows a device 10 for monitoring a temporal trigger stability. In this embodiment, the device 10 is designed as an ECG module. ECG signals can be received via an interface 12 and signals can be transmitted to other devices or devices without the need for a particular coupling. The interface can thus enable a communicative coupling with an extracorporeal circulatory support system or an extracorporeal circulatory support device. It can also be designed together with an interface of a control unit.

The device 10 can optionally be formed as an ECG device, which can be integrated into a control unit or attached to it, so that a control unit of an extracorporeal circulatory support device can be provided, which can be used independently of the provision of other components and is compact in design. In some embodiments, the device 10 can be integrated into a single housing of a system for an extracorporeal circulatory support, for example in a sensor box.

An ECG signal 14 of a circulatory-supported patient can be received via the interface 12. The corresponding data can be temporarily stored in a working memory and optionally stored in a storage medium 18 of the device 10. The ECG signal 14 is forwarded from the interface 12 or via the working memory to an evaluation unit 16. The evaluation unit 16 is also communicatively connected to the storage medium 18. It is configured to receive or read out at least one pathological ECG signature 20 with evaluated ECG signals or corresponding threshold values or parameter values corresponding to a respective pathology and to compare the ECG signal 14 with the at least one ECG signature 20.

The ECG signature 20 can be selected according to the best possible superposition, for example with the statistically greatest possible superposition of an averaged P-R time interval, QRS time interval and/or R-T time interval of several ECG signals 14. This can serve as a basis for subsequent cardiac cycles when determining a trigger signal 22 and/or when detecting the P-wave, R-wave and/or T-wave.

In other words, suitable threshold values and/or frequency ranges for the respective time interval from the pathological ECG signature 20 can be used and form the basis for further processing and use of the ECG signal 14. For example, the selected pathological ECG signature can be used to determine or select a trigger signal 22 from the ECG signal 14 for subsequent cardiac cycles, which can be used to control and synchronize a blood pump of an extracorporeal circulatory support system. For this purpose, the trigger signal 22 can be forwarded to the interface 12 or be provided there. At this interface 12, the trigger signal 22 can then be retrieved or received by a control unit, for example.

If one or more threshold values for subsequent cardiac cycles are exceeded or if threshold value ranges specified on the basis of the pathological ECG signature 20 are not (regularly) adhered to or met, the evaluation unit 16 can again perform a comparison with data records stored “offline” in the storage medium 18 and thus with further pathological ECG signatures 20 in order to optimize the procedure again and further improve the trigger stability. Such a process can be carried out periodically. However, it can be performed continuously so that any changes in the ECG signal 14 can be recognized and taken into account.

Where applicable, all the individual features shown in the exemplary embodiments may be combined and/or exchanged without departing from the scope of the invention.

### LIST OF REFERENCE NUMERALS

10 Device
12 Interface
14 ECG signal
16 Evaluation unit
18 Storage medium
20 Pathological ECG signature
22 Trigger signal

1-32. (canceled)

33. A method of monitoring and improving a temporal trigger stability of an extracorporeal circulatory support, the method comprising:
receiving an ECG signal of a supported patient over a predefined period of time;
determining P-waves, R-waves, and T-waves from the received ECG signal; and
determining a predefined trigger signal from the ECG signal of a current cardiac cycle, taking into account at least one of: (i) at least one P-wave determined from the received ECG signal and (ii) at least one T-wave determined from the received ECG signal.

34. The method according to claim 33, wherein the at least one P-wave and/or the at least one T-wave is determined for a current cardiac cycle.

35. The method according to claim 33, wherein the at least one P-wave and/or the at least one T-wave is determined for at least one previous cardiac cycle.

36. The method according to claim 33, wherein the at least one P-wave is detected at least for a current cardiac cycle and the at least one T-wave is detected at least for a directly preceding cardiac cycle.

37. The method according to claim 33, wherein the predefined trigger signal is determined based on a P-R time interval between the P-wave and the R-wave of a respective cardiac cycle.

38. The method according to claim 37, wherein determination of the predefined trigger signal is adjusted when a threshold value predefined for the P-R time interval is exceeded.

39. The method according to claim 38, wherein adjustment of the determination of the predefined trigger signal is a change in the predefined trigger signal from the R-wave to the P-wave or from the P-wave to the R-wave.

40. The method according to claim 37, wherein determination of at least one of the P-wave, R-wave and T-wave for a current cardiac cycle is adjusted, if a threshold value for the P-R time interval is exceeded for at least one preceding respective cardiac cycle.

41. The method according to claim 37, wherein the P-R time interval is averaged for at least two cardiac cycles.

42. The method according to claim 37, wherein the P-R time interval is compared for at least two successive cardiac cycles, and wherein determination of the predefined trigger signal is adjusted upon a successive extension of the P-R time interval.

43. The method according to claim 33, wherein the predefined trigger signal is determined based on an R-T time interval between the R-wave and the T-wave of a respective cardiac cycle.

44. The method according to claim 43, wherein determination of the predefined trigger signal is adjusted when a threshold value for the R-T time interval is exceeded.

45. The method according to claim 43, wherein determination of at least one of the P-wave, R-wave and T-wave for a current cardiac cycle is adjusted, if a threshold value for the R-T time interval is exceeded for at least one preceding respective cardiac cycle.

46. The method according to claim 43, wherein the R-T time intervals of at least two cardiac cycles are averaged.

47. The method according to claim 33, wherein determination of at least one of: the predefined trigger signal, the P-wave, the R-wave and the T-wave for a current cardiac cycle is adjusted when an amplitude of the T-wave for at least one previous preceding cycle comprises a negative value or is negative in total.

48. The method according to claim 33, wherein the predefined trigger signal is determined based on a QRS time interval between a Q-wave and an S-wave of a respective cardiac cycle,
wherein determination of at least one of: the predefined trigger signal, the P-wave, the R-wave, and the T-wave is adjusted when a predefined threshold value for the QRS time interval is exceeded for at least one preceding respective cardiac cycle.

49. The method according to claim 33, wherein at least one of the predefined trigger signal and a threshold value for one or more time intervals within a respective cardiac cycle is based on a pathological ECG signature.

50. The method according to claim 49, wherein the pathological ECG signature is predefined or is determined for the current cardiac cycle based on time intervals recorded from preceding cardiac cycles.

51. The method according to claim 50, wherein the time intervals detected from the preceding cardiac cycles are compared with an “offline” evaluated data set with ECG signals from supported patients with a corresponding patient pathology, and wherein the pathological ECG signature is predefined based on a best-fit match with an ECG signal from the data set.

52. The method according to claim 51, wherein for each known pathology and for a respective time interval at least one of: a time interval range, a frequency range, an amplitude range, and the predefined trigger signal is predefined.

53. The method according to claim 33, wherein the at least one of the P-wave, the R-wave and the T-wave is detected by means of a bandpass filter, wherein at least one of: (i) a frequency range for the bandpass filter is between 5 Hz and 20 Hz for the P-wave, (ii) the frequency range for the bandpass filter is between 10 Hz and 25 Hz for the R-wave, and (iii) the frequency range for the bandpass filter is between 1 Hz and 5 Hz for the T-wave.

54. The method according to claim 33, wherein the predefined trigger signal is the R-wave, and wherein the P-wave or the T-wave is selected as a further trigger signal to be determined by taking into account the at least one P-wave determined from the ECG signal and/or the at least one T-wave determined from the ECG signal.

55. The method according to claim 33, wherein based on the determined predefined trigger signal, a signal for the extracorporeal circulatory support is output with a delay time, wherein the delay time is predefined taking into account the at least one P-wave and/or the at least one T-wave.

56. The method according to claim 55, wherein the delay time for a current cardiac cycle is determined based on a detected QT time from at least one previous cardiac cycle, and wherein the QT time is taken into account based on a heart rate determined from the ECG signal.

57. A device for monitoring and improving a temporal trigger stability of an extracorporeal circulatory support, the device comprising:
an interface for receiving an ECG signal of a supported patient over a predefined period of time, and
an evaluation unit which is configured to determine P-waves, R-waves and T-waves from the received ECG signal, and to determine a predefined trigger signal from the ECG signal of a current cardiac cycle, taking into account at least one determined P wave and/or at least one determined T wave.

58. The device according to claim 57, configured to carry out a method comprising:
receiving the ECG signal of the supported patient over the predefined period of time;
determining the P-waves, the R-waves, and the T-waves from the received ECG signal; and
determining the predefined trigger signal from the ECG signal of the current cardiac cycle, taking into account at least one of: (i) the at least one P-wave determined from the received ECG signal and (ii) the at least one T-wave determined from the received ECG signal.
